(19) United States
(12) Patent Application Publication
(10) Pub. No.: US 2025/0351807 A1
(43) Pub. Date: Nov. 20, 2025

(54) WILD-DERIVED MOUSE MODELS OF ALZHEIMER’S DISEASE

(71) Applicant: The Jackson Laboratory, Bar Harbor, ME (US)

(72) Inventors: Kristen Onos, Bar Harbor, ME (US); Gareth Howell, Bar Harbor, ME (US); Michael Sasner, Bar Harbor, ME (US); Gregory Carter, Bar Harbor, ME (US)

(73) Assignee: The Jackson Laboratory, Bar Harbor, ME (US)

(21) Appl. No.: 18/844,781

(22) PCT Filed: Mar. 9, 2023
(86) PCT No.: PCT/US2023/064031

§ 371 Date: Sep. 6, 2024

Related U.S. Application Data
(60) Provisional application No. 63/318,313, filed on Mar. 9, 2022.

(51) Int. Cl. A01K 67/0278; C07K 14/47

(52) U.S. Cl. CPC A01K 67/0278; C07K 14/4711; A01K 2207/15; A01K 2217/052; A01K 2227/105; A01K 2267/0312

(57) ABSTRACT

The present disclosure provides wild-derived mouse models that comprise a nucleic acid encoding a human amyloid precursor protein (APP), a nucleic acid encoding a mutated human presenilin (1) protein (PSEN1), and in some embodiments, a human apolipoprotein E (APOE), or human amyloid beta and human tau. These mouse models are useful, for example, for Alzheimer's disease studies.

### RELATED APPLICATION

This application claims the benefit under 35 U.S.C. § 119 (e) of U.S. provisional application No. 63/318,313, filed Mar. 9, 2022, which is incorporated by reference herein in its entirety.

### REFERENCE TO AN ELECTRONIC SEQUENCE LISTING

The contents of the electronic sequence listing (J022770116WO00-SEQ-EAS.xml; Size: 4,984 bytes; and Date of Creation: Mar. 9, 2023) is herein incorporated by reference in its entirety.

### BACKGROUND

Transgenic mouse models expressing human amyloid precursor protein (APP) with or without the expression of human presenilin 1 (PSEN1) have been used extensively to study Alzheimer's disease (AD) in vivo to gain a better understanding of pathogenesis of the disease in human patients. Nevertheless, such models often inadequately recapitulate the widespread neurodegeneration and regional brain atrophy that occurs in AD (Drummond et al., Acta Neuropathol. 2017 February; 133 (2): 155-175). Additionally, such models have been made on limited background strains, such as C57BL6/J. These transgenic mouse strains have exhibited a large burden of parenchymal amyloid deposits that are likely greater than those seen in human patients. In general, another key hallmark, neuroinflammation, has been limited to responses to specific to parenchymal plaques. Due to all of the reasons mentioned, existing transgenic mouse models expressing APP are limited in their capacity to recapitulate human-relevant AD pathology.

### SUMMARY

The present disclosure provides, in some aspects, improved wild-derived “humanized” mouse models of Alzheimer's disease (AD). As is known in the art, mice that express a human gene/protein are often referred to as “humanized” mice. It should be understood that a human gene comprises human nucleic acid sequence(s) that encodes a human protein or a human protein domain.

In some embodiments, a wild-derived humanized mouse model of AD expresses a human amyloid precursor protein (APP), expresses a mutated human presenilin 1 protein (PSEN1, also abbreviated as PSEN1), and expresses (a) a human apolipoprotein (APOE) (e.g., a human apolipoprotein E4, E3, or E2) or (b) a human amyloid beta and/or a human tau. Modeling AD on a wild-derived background permits a platform for studying interactions with amyloid on a more human-relevant, genetically diverse background. This background also allows the exploration of a range of neuroinflammatory responses.

The wild-derived humanized mouse models provided herein are based, at least in part, on the theory that studying the pathogenesis of AD by modulating known genetic risk factors in a genetically diverse mouse background results in a more clinically relevant model. This theory was tested by introducing human risk alleles APOE4 and human amyloid beta and/or human tau to a wild-derived humanized mouse model expressing human APP and a mutated human PSEN1. First, a WSB.APOE4 mouse model was generated. The WSB.APP/PSEN1 model was then crossed to the WSB.APOE4 mouse model to generate a novel wild-derived humanized mouse model expressing a human APP, a mutated human PSEN1, and a human APOE4 (the “WSB.APP/PSEN1/APOE4” model). It was surprising shown that wild-derived humanized mouse models of AD that comprise transgenic amyloid mutations lead to neurodegeneration in both the cortex and hippocampus of female mice at as early as 8 months.

Thus, some aspects of the present disclosure provide a wild-derived humanized mouse comprising in its genome a nucleic acid encoding a human amyloid precursor protein (APP), a nucleic acid encoding a mutated human presenilin 1 protein (PSEN1), and a nucleic acid encoding a human apolipoprotein E.

In some embodiments, the human apolipoprotein E is a human apolipoprotein E4 (APOE4).

Other aspects of the present disclosure provide a wild-derived humanized mouse comprising in its genome a nucleic acid encoding a human amyloid precursor protein (APP), a nucleic acid encoding a mutated human presenilin 1 protein (PSEN1), a nucleic acid encoding a human amyloid beta, and a nucleic acid encoding a human tau.

In some embodiments, the nucleic acid encoding a human APP is a chimeric nucleic acid comprising mouse and human coding sequences.

In some embodiments, the chimeric nucleic acid comprises a human coding sequence in the A-beta domain of a mouse APP coding sequence.

In some embodiments, the chimeric nucleic acid encodes human mutations K595N and M596L, relative to a human APP comprising the amino acid sequence of SEQ ID NO: 1.

In some embodiments, the nucleic acid encoding a human APP is an APPswe transgene.

In some embodiments, the nucleic encoding a mutated PSEN1 comprises a human PSEN1 coding sequence that comprises a deletion in exon 9.

In some embodiments, the nucleic acid encoding a mutated PSEN1 is a PSEN1de9 transgene.

In some embodiments, the mouse comprises in its genome Tg(APPswe,PSEN1de9)85Dbo transgene insertion.

In some embodiments, the mouse expresses the human APP, the human PSEN1, and the human apolipoprotein E, optionally human APOE4.

In some embodiments, the mouse expresses the human amyloid beta and the human tau.

In some embodiments, the mouse has a genetic background selected from WSB/EiJ, CAST/EiJ, and PWK/PhJ.

In some embodiments, the mouse has at least one characteristic of early-onset Alzheimer's disease, for example, at least one characteristic of early-onset Alzheimer's disease is selected from neurodegeneration, cognitive deficit, and increased neuroinflammation in the brain, relative to a control.

In some embodiments, the mouse does not develop a tumor or have a measurable tumor burden.

In some embodiments, the mouse is at least a year old.

Some aspects of the present disclosure provide a wild-derived humanized WSB mouse comprising in its genome a APPswe transgene, and a PSENde9 transgene, and a gene encoding human apolipoprotein E, optionally human apolipoprotein E4 (APOE4).

Other aspects of the present disclosure provide a wild-derived humanized mouse comprising in its genome a nucleic acid encoding human apolipoprotein E, optionally a human apolipoprotein E4 (APOE4).

Yet other aspects of the present disclosure provide a wild-derived humanized mouse comprising in its genome a humanized amyloid beta and a humanized tau.

Still other aspects of the present disclosure provide a cell from the mouse of any one of the preceding paragraphs.

Further aspects of the present disclosure provide a mouse comprising a cell having the same genotype of a cell from the mouse of any one of the preceding paragraphs.

Some aspects of the present disclosure provide a progeny mouse of the mouse of any one of the preceding paragraphs.

Some aspects of the present disclosure provide a method comprising producing the mouse of any one of the preceding paragraphs.

### BRIEF DESCRIPTION OF THE DRAWINGS

FIGS. 1A and 1B depict immunohistochemistry and corresponding quantification of brain tissue from 8-month-old female mice of WSB, WSB.APOE4, WSB.APP/PSEN1, and WSB.APP/PSEN1/APOE4 genotypes. FIG. 1A depicts brain tissues strained with DAPI and the antibody NEUN on multiple coronal brain sections ranging from anterior, mid and posterior areas of the brain. FIG. 1B depicts the amount of NEUN measured in the traced cortical regions averaged across each genotype (top) and a comparison of cortical surface area (lower).

FIG. 2 is a graph depicting a comparison of cortical surface area of brain tissue from 4-month-old female mice of WSB, WSB.APOE4, WSB.APP/PSEN1, and WSB.APP/PSEN1/APOE4 genotypes.

### DETAILED DESCRIPTION

Alzheimer's disease (AD) is the most common cause of dementia. AD affects 35 million people today and its worldwide prevalence is expected to reach 115 million by 2050 due to aging of the population. AD progresses through three stages: preclinical, mild cognitive impairment (MCI), and dementia. Humans with MCI have cognitive deficits but no functional impairments, while humans with dementia exhibit a decline of two or more cognitive domains, which has gradually progressed to the point that functioning at work or daily activities is impaired. Pathologically, AD diagnosis in humans is based on protein aggregates in the brain including amyloid plaques composed of amyloid-beta (Aβ) peptides and neurofibrillary tangles (NFTs) composed of hyperphosphorylated tau. In humans, early spatial distribution of plaque pathology, including plaque pathology occurring first in the hippocampus, correlates strongly with diagnosis of dementia.

Mouse models of AD are limited in that none of the existing models have exhibited the full range of clinical and pathological features of AD, including cognitive and behavioral deficits, amyloid plaques, neurofibrillary tangles, gliosis, synapse loss, axonopathy, neuron loss and neurodegeneration. Importantly, different mouse models provide varying degrees of AD phenotypes. For example, phenotypes such as cognitive deficits and amyloid plagues are observed in almost all of the mouse models of AD, however human pathology of AD has yet to be recapitulated. In a B6.APP/PSEN1 mouse model, for example, hippocampal and robust cortical plaque deposition is seen at an early timepoint, which is in contrast to human pathology in which plaques are primarily limited to the hippocampus. Unlike the B6.APP/PSEN1 mouse model, the mouse models of the present disclosure, which model AD on a wild-derived background, exhibit severe neurodegeneration, which more closely resembles the human AD pathology.

In some embodiments, the present disclosure provides wild-derived humanized mouse models (e.g., WSB/EiJ mouse models) that comprise a human amyloid precursor protein (APP), a mutated human presenilin 1 protein (PSEN1), and a human apolipoprotein E (e.g., a human apolipoprotein E4, E3, or E2). In some embodiments, the present disclosure provides wild-derived humanized mouse models (e.g., WSB/EiJ mouse models) that comprise a human APP, a mutated human PSEN1, and a human amyloid beta and/or a human tau.

### Parental Strains and Alleles

In some embodiments, the mouse models provided herein are generated on a wild type-derived genetic background, such as the WSB background. In some embodiments, the mouse models provided herein are generated using a WSB/EiJ strains, such as the WSB.Cg-Tg(APPswe,PSEN1dE9)85Dbo/How strain (WSB.APP/PS1). WSB.APP/PS1 are double transgenic mice expressing a chimeric mouse/human amyloid precursor protein (Mo/HuAPP695swe) and a mutant human presenilin 1 (PS1-dE9), both directed to CNS neurons. Both mutations are associated with early-onset Alzheimer's disease. The “humanized” Mo/HuAPP695swe transgene allows the mice to secrete a human A-beta peptide. Both the transgenic peptide and holoprotein can be detected by antibodies specific for human sequence within this region (Signet Laboratories' monoclonal 6E10 antibody). The included Swedish mutations (K595N/M596L) elevate the amount of A-beta produced from the transgene by favoring processing through the beta-secretase pathway. WSB.APP/PS1 female hemizygotes exhibit increased loss of cortical region and CA1 NEUN+DAPI+ (hippocampal) neurons, as well as impaired short-term memory, compared to controls. Cortical plaques are fewer in number compared to B6.APP/PS1 mice. WSB.APP/PS1 males exhibit a decreased number of hippocampal plaques (compared to B6.APP/PS1 controls), while WSB.APP/PS1 females do not. WSB.APP1/PS1 mice were generated by backcrossing B6.Cg-Tg(APPswe,PSEN1dE9)85Dbo/Mmjax (JAX Stock No. 34832-JAX) mice to WSB/EiJ (JAX Stock No. 001145) mice for 12 generations.

In some embodiments, the mouse models provided herein are generated on a wild type-derived genetic background, such as the PWK background. In some embodiments, the mouse models provided herein are generated using a PWK/PhJ strain, such as the PWK.Cg-Tg(APPswe,PSEN1dE9)85Dbo/How strain (PWK.APP/PS1). PWK.APP1/PS1 mice were generated by backcrossing B6.Cg-Tg(APPswe,PSEN1dE9)85Dbo/Mmjax (Stock No. 34832-JAX) mice to PWK/PhJ (Stock No. 003715) mice for 11 generations.

In some embodiments, the mouse models provided herein are generated on a wild type-derived genetic background, such as the CAST background. In some embodiments, the mouse models provided herein are generated using a CAST/EiJ strain. This strain may be backcrossed, for example, to B6.Cg-Tg(APPswe,PSEN1dE9)85Dbo/Mmjax (JAX Stock No. 34832-JAX) for multiple generations.

### Amyloid Precursor Protein

Amyloid precursor protein is a single-pass (type-I) transmembrane precursor protein that is a cleaved into amyloid beta (Aβ), the primary component of amyloid plaques, and is associated with at least one characteristic of early-onset Alzheimer's disease. Knocking-in chimeric mouse/human amyloid precursor protein can lead to secretion of human amyloid-β (Aβ) peptide. In some embodiments, a mouse model comprises a chimeric nucleic acid that comprises a human coding sequence in the A-beta domain of a mouse APP coding sequence. In some embodiments, the chimeric nucleic acid encodes human Swedish mutations K595N and M596L, relative to a human APP comprising the amino acid sequence of SEQ ID NO: 1. The included Swedish mutations (K595N and M596L) elevate the amount of A-beta produced from the transgene by favoring processing through the beta-secretase pathway (Shin et al. 2010). In some embodiments, the chimeric nucleic acid is the APPswe transgene, which encodes a chimeric amyloid beta (A4) precursor protein comprising the Swedish mutations K595N and M596L. See, e.g., Borchelt, David R., et al. Neuron 1996; 17 (5): 1005-1013; JAX Stock No. 025970).

In some embodiments, a human amyloid precursor protein comprises a sequence that is at least 80%, at least 85%, at least 90%, at least 95%, at least 96%, at least 97%, at least 98%, at least 99%, or 100% identical to the sequence of SEQ ID NO: 1:

(SEQ ID NO: 1)
MLPGLALLLLAAWTARALEVPTDGNAGLLAEPQIAMFCGRLNMHMNVQNG
KWDSDPSGTKTCIDTKEGILQYCQEVYPELQITNVVEANQPVTIQNWCKR
GRKQCKTHPHFVIPYRCLVGEFVSDALLVPDKCKFLHQERMDVCETHLHW
HTVAKETCSEKSTNLHDYGMLLPCGIDKFRGVEFVCCPLAEESDNVDSAD
AEEDDSDVWWGGADTDYADGSEDKVVEVAEEEEVAEVEEEEADDDEDDED
GDEVEEEAEEPYEEATERTTSIATTTTTTTESVEEVVREVCSEQAETGPC
RAMISRWYFDVTEGKCAPFFYGGCGGNRNNFDTEEYCMAVCGSAMSQSLL
KTTQEPLARDPVKLPTTAASTPDAVDKYLETPGDENEHAHFQKAKERLEA
KHRERMSQVMREWEEAERQAKNLPKADKKAVIQHFQEKVESLEQEAANER
QQLVETHMARVEAMLNDRRRLALENYITALQAVPPRPRHVFNMLKKYVRA
EQKDRQHTLKHFEHVRMVDPKKAAQIRSQVMTHLRVIYERMNQSLSLLYN
VPAVAEEIQDEVDELLQKEQNYSDDVLANMISEPRISYGNDALMPSLTET
KTTVELLPVNGEFSLDDLQPWHSFGADSVPANTENEVEPVDARPAADRGL
TTRPGSGLTNIKTEEISEVKMDAEFRHDSGYEVHHQKLVFFAEDVGSNKG
AIIGLMVGGVVIATVIVITLVMLKKKQYTSIHHGVVEVDAAVTPEERHLS
KMQQNGYENPTYKFFEQMQN.

### Presenilin 1

Presenilin 1 PSEN1 is a subunit of gamma- (γ-)secretase complex that is involved in the cleavage of APP resulting in the amyloid-β peptide. Mouse models that express mutated human presenilin 1 and a human APP transgene are associated with at least one characteristic of early-onset Alzheimer's disease. In some embodiments, a nucleic acid encoding a mutated PSEN1 comprises a human PSEN1 coding sequence that comprises a deletion in exon 9 (DeltaE9). In some embodiments, the nucleic acid is the PSEN1de9 transgene. In some embodiments, the PSEN1de9 transgene is the Tg(APPswe,PSEN1de9)85Dbo transgene insertion See, e.g., Borchelt et al. 1996; JAX Stock No. 025970.

In some embodiments, a human presenilin 1 protein comprises a sequence that is at least 80%, at least 85%, at least 90%, at least 95%, at least 96%, at least 97%, at least 98%, at least 99%, or 100% identical to the sequence of SEQ ID NO: 2:

(SEQ ID NO: 2)
MTELPAPLSYFQNAQMSEDNHLSNTVRSQNDNRERQEHNDRRSLGHPEPL
SNGRPQGNSRQVVEQDEEEDEELTLKYGAKHVIMLFVPVTLCMVVVVATI
KSVSFYTRKDGQLIYTPFTEDTETVGQRALHSILNAAIMISVIVVMTILL
VVLYKYRCYKVIHAWLIISSLLLLFFFSFIYLGEVFKTYNVAVDYITVAL
LIWNFGVVGMISIHWKGPLRLQQAYLIMISALMALVFIKYLPEWTAWLIL
AVISVYDLVAVLCPKGPLRMLVETAQERNETLFPALIYSSTMVWLVNMAE
GDPEAQRRVSKNSKYNAESTERESQDTVAENDDGGFSEEWEAQRDSHLGP
HRSTPESRAAVQELSSSILAGEDPEERGVKLGLGDFIFYSVLVGKASATA
SGDWNTTIACFVAILIGLCLTLLLLAIFKKALPALPISITFGLVFYFATD
YLVQPFMDQLAFHQFYI

### Apolipoprotein E (APOE)

The APOE gene provides instructions for making apolipoprotein E. This protein combines with fats (lipids) in the body to form molecules called lipoproteins. Lipoproteins are responsible for packaging cholesterol and other fats and carrying them through the bloodstream. Maintaining normal levels of cholesterol is essential for the prevention of disorders that affect the heart and blood vessels (cardiovascular diseases), including heart attack and stroke.

There are at least three slightly different versions (alleles) of the APOE gene. The major alleles are APOE2, APOE3, and APOE4. The most common allele is APOE3, which is found in more than half of the general population. In some embodiments, a nucleic acid encodes a human APOE2, APOE3, or APOE4.

Apolipoprotein E4 (APOE4) is a lipo-binding protein that is involved lipoprotein metabolism and is one of the largest known genetic risk factors for late-onset sporadic AD. In some embodiments, a nucleic acid encodes a human APOE4.

In some embodiments, the human APOE4 protein comprises a sequence that is at least 80%, at least 85%, at least 90%, at least 95%, at least 96%, at least 97%, at least 98%, at least 99%, or 100% identical to the sequence of SEQ ID NO: 3:

(SEQ ID NO: 3)
MKVLWAALLVTFLAGCQAKVEQAVETEPEPELRQQTEWQSGQRWELALGR
FWDYLRWVQTLSEQVAEVRAKLEEQAQQIRLQAEAFQARLKSWFEPLVED
MQRQWAGLVEKVQAAVGTSAAPVPSDNH.

### Amyloid Beta

Amyloid beta refers to the peptides of 36-43 amino acids that are the main component of the amyloid plaques found in the brains of people with AD. The peptides derive from the amyloid precursor protein (APP), which is cleaved by beta secretase and gamma secretase to yield Aβ in a cholesterol dependent process and substrate presentation. In some embodiments, a nucleic acid encodes a human amyloid beta.

### Tau

Tau refers to soluble protein isoforms produced by alternative splicing from the gene MAPT (microtubule-associated protein tau) and is associated with pathologies and dementias of the nervous system such as AD. In some embodiments, a nucleic acid encodes a human tau.

### Assessment of Neurodegeneration and Other Symptoms of Alzheimer's Disease

Alzheimer's disease is a brain disorder that slowly destroys memory and thinking skills, and, eventually, the ability to carry out the simplest tasks. In most people with Alzheimer's, symptoms first appear later in life. Estimates vary, but experts suggest that more than 6 million Americans, most of them age 65 or older, may have dementia caused by Alzheimer's disease. Alzheimer's disease is currently ranked as the seventh leading cause of death in the United States and is the most common cause of dementia among older adults.

Dementia is the loss of cognitive functioning—thinking, remembering, and reasoning—and behavioral abilities to such an extent that it interferes with a person's daily life and activities. Dementia ranges in severity from the mildest stage, when it is just beginning to affect a person's functioning, to the most severe stage, when the person must depend completely on others for help with basic activities of daily living. The causes of dementia can vary, depending on the types of brain changes that may be taking place. Other dementias include Lewy body dementia, frontotemporal disorders, and vascular dementia. It is common for people to have mixed dementia—a combination of two or more types of dementia. For example, some people have both Alzheimer's disease and vascular dementia.

As used herein, characteristics of early-onset Alzheimer's disease include, but are not limited to neurodegeneration, vascular deficit, mitochondrial dysfunction, cognitive deficit, amyloid plaque deposition, cortical plaque deposition, and neuroinflammation in the brain.

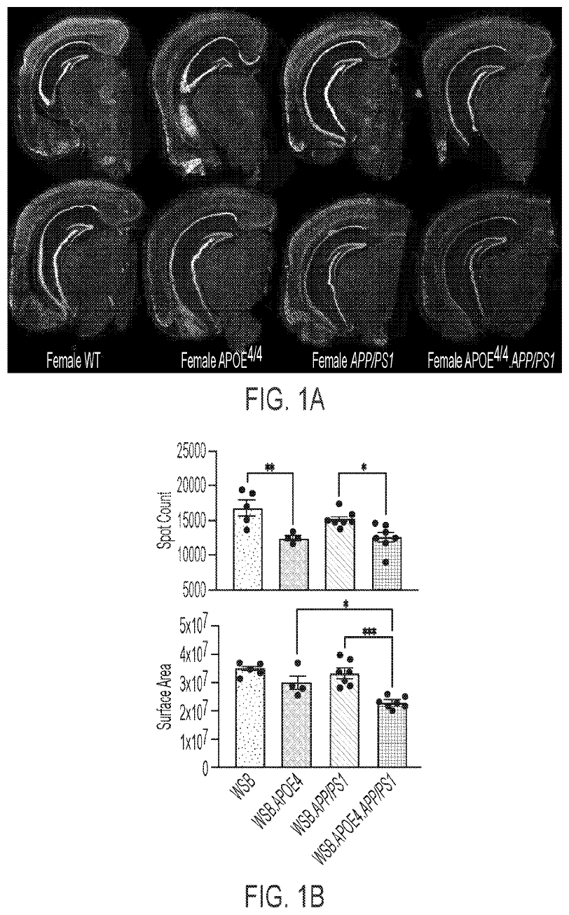

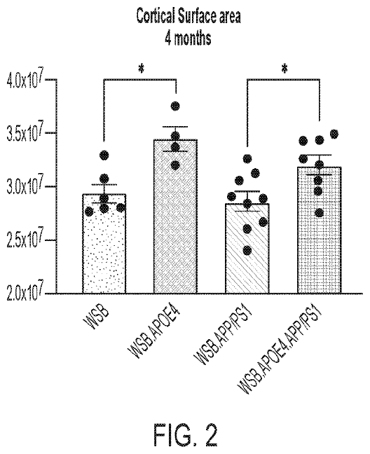

In some embodiments, a wild-derived humanized mouse model of the present invention comprising in its genome a nucleic acid encoding a human amyloid precursor protein (APP), a nucleic acid encoding a mutated human presenilin 1 protein (PSEN1), and a nucleic acid encoding a human apolipoprotein E (e.g., a human apolipoprotein E4, E3, or E2) (e.g., a WSB.APP/PSEN1/APOE4 mouse and/or WSB.APOE4). Surprisingly, this mouse has at least 20%, at least 30%, or at least 40% greater, or about 20-40% greater, neurodegeneration relative to a control mouse (e.g., a WSB mouse, such as a WSB.APP/PSEN1 mouse, or a C57BL6/J mouse) of the same age (see FIGS. 1A-1B and FIG. 2). As used herein, neurodegeneration refers to the reduction or depletion of neurons. Neurodegeneration may be measured using immunofluorescent staining of NEUN, a surrogate for neurons, in the regions of a mouse brain. Immunofluorescent staining methods, which are well-known in the art, are contemplated herein. Positive staining for NEUN, indicating neurons, may be present in the coronal brain sections ranging from anterior, mid and posterior areas of the brain of a mouse brain of the present disclosure. Total immunofluorescent staining of neurodegeneration in a region of the wild-derived mouse brain can be compared relative to the total immunofluorescent staining in a different region of the same wild-derived mouse brain. Total immunofluorescent staining of neurodegeneration in a mouse brain can also be compared relative to the total immunofluorescent staining of neurodegeneration in a control mouse brain.

In some embodiments, in a wild-derived humanized mouse model of the present disclosure, the neurodegeneration in a brain region may be increased by at least 5%, at least 10%, at least 20%, at least 30%, at least 35%, at least 40%, at least 45%, at least 50%, at least 55%, at least 60%, at least 65%, or at least 70% relative to the neurodegeneration in a brain region of a control mouse of the same age. In some embodiments, neurodegeneration may be assessed using a spot count immunohistochemical assay. In some embodiments, in a wild-derived humanized mouse model, the neurodegeneration in a brain region may be increased by at least 20% relative to the neurodegeneration in a brain region of a control mouse of the same age. In some embodiments, in a wild-derived humanized mouse model, the neurodegeneration in a brain region may be increased by about 20% relative to the neurodegeneration in a brain region of a control mouse of the same age. In other embodiments, neurodegeneration may be assessed based on surface area. In some embodiments, in a wild-derived humanized mouse model, the neurodegeneration in a brain region may be increased by at least 35% or at least 40% relative to the neurodegeneration in a brain region of a control mouse of the same age. In some embodiments, in a wild-derived humanized mouse model, the neurodegeneration in a brain region may be increased by about 35% or about 40% relative to the neurodegeneration in a brain region of a control mouse of the same age.

In some embodiments, a wild-derived humanized mouse model of the present disclosure has greater vascular deficit relative to a control mouse (e.g., a WSB mouse, such as a WSB.APP/PSEN1 mouse, or a C57BL6/J mouse) of the same age. As used herein, vascular deficit refers any reduction is vascular function known to be associated with AD. Vascular deficit may be measured using immunofluorescent staining of known vascular markers. Immunofluorescent staining methods, which are well-known in the art, are contemplated herein. Total immunofluorescent staining of vascular deficit in a region of the wild-derived humanized mouse brain can be compared relative to the total immunofluorescent staining in a different region of the same wild-derived humanized mouse brain. Total immunofluorescent staining of vascular deficit in a mouse brain can also be compared relative to the total immunofluorescent staining of vascular deficit in a control mouse brain.

In some embodiments, in a wild-derived humanized mouse model of the present disclosure, the vascular deficit in a brain region may be increased by at least 5%, at least 10%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to the vascular deficit in a brain region of a control mouse of the same age.

In some embodiments, a wild-derived humanized mouse model of the present disclosure has greater mitochondrial dysfunction relative to a control mouse (e.g., a WSB mouse, such as a WSB.APP/PSEN1 mouse, or a C57BL6/J mouse) of the same age. As used herein, mitochondrial dysfunction refers any reduction in mitochondrial function known to be associated with AD. Mitochondrial dysfunction may be measured using immunofluorescent staining of known mitochondrial markers. Immunofluorescent staining methods, which are well-known in the art, are contemplated herein. Total immunofluorescent staining of mitochondrial dysfunction in a region of the wild-derived humanized mouse brain can be compared relative to the total immunofluorescent staining in a different region of the same wild-derived humanized mouse brain. Total immunofluorescent staining of mitochondrial dysfunction in a mouse brain can also be compared relative to the total immunofluorescent staining of mitochondrial dysfunction in a control mouse brain.

In some embodiments, in a wild-derived humanized mouse model, the mitochondrial dysfunction in a brain region may be increased by at least 5%, at least 10%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to the mitochondrial dysfunction in a brain region of a control mouse of the same age.

In some embodiments, a wild-derived humanized mouse model of the present disclosure has greater cognitive deficits relative to a control mouse (e.g., a WSB mouse, such as a WSB.APP/PSEN1 mouse, or a C57BL6/J mouse) of the same age. As used herein, cognitive deficits are used to describe the impairment of different domains of cognition and is used interchangeably with the term cognitive impairment. Cognitive deficits in a mouse of the present disclosure may be measured according to, but not limited by, any of the following behavioral assays: Y-maze measures of working and short-term memory, hole-board, open field, and Touchscreen.

The Y-maze is used to evaluate short term memory in mice. Spontaneous alternation, a measure of spatial working memory, can be assessed by allowing mice to explore all three arms of the maze and is driven by an innate curiosity of rodents to explore previously unvisited areas. A mouse with intact working memory, and hence intact prefrontal cortical functions, will remember the arms previously visited and show a tendency to enter a less recently visited arm. Spatial reference memory, which is underlined by the hippocampus, can also be tested by placing the test mice into the Y-maze with one arm closed off during training. This test is based on the spontaneous tendency of mice to spend more time exploring a novel arm than a familiar one. In some embodiments, a wild-derived humanized mouse model of the present disclosure may spend more equal or less time exploring a novel arm relative to a familiar object when compared with a control mouse.

The hole-board task is used to measure exploratory behavior, locomotor activity and cognitive function in mice. The test is based on a mouse's natural curiosity and attraction for novelty. A hole-board consists of a small square arena with an extractable platform as floor, which has a set of equally spaced circular holes on its surface. Mice spontaneously approach the holes and explore them by briefly inserting the snout inside, a behavior defined as nose-poking (or head-dipping). If, after a period of time, mice are re-exposed to the hole-board, the novelty of the holes decreases. Animals with an intact long-term memory will show a reduction of the frequency of nose-poking into the holes. In some embodiments, a wild-derived humanized mouse model of the present disclosure may have increased nose-poking events, relative to a control mouse that would avoid nose-poking holes that were previously explored by said mouse.

The Open Field task is a simple sensorimotor test used to determine general activity levels, gross locomotor activity, and exploration habits in rodent models of CNS disorders. Assessment takes place in a square, white Plexiglas box. The animal is placed in the arena and allowed to freely move about for 10 minutes while being recorded by an overhead camera. The footage is then analyzed by an automated tracking system for the following parameters: distance moved, velocity, and time spent in pre-defined zones. In some embodiments, a wild-derived humanized mouse model of the present disclosure may exhibit different levels of activity levels, gross locomotor activity, and exploration habits relative to a control mouse.

The touchscreen task is a method to perform simple and complex cognitive neuroscience measurements in mice. The touchscreen task uses an automated touchscreen platform, in which a remarkable diversity of cognitive functions may be tested in mice. In some embodiments, a wild-derived humanized mouse model of the present disclosure may exhibit lower cognitive functions relative to a control mouse.

In some embodiments a wild-derived humanized mouse model of the present disclosure has increased amyloid plaque deposition in the hippocampal region of the brain relative to the cortical region of the brain. As used herein, amyloid plaque deposition refers to the A-beta protein deposition, which accumulates progressively and forms plaque-like lesions throughout the span of the mouse. Amyloid plaque deposition may be measured using immunofluorescent staining of amyloid precursor protein in the cortical and/or hippocampal regions of a mouse brain. Immunofluorescent staining methods, which are well-known in the art, are contemplated herein. Positive staining for amyloid precursor protein, indicating amyloid plaque deposition, may be present in the cortical or the hippocampal region, or both the cortical and hippocampal regions of a mouse brain of the present disclosure. Total immunofluorescent staining of amyloid plaque deposition in the cortical region can be compared relative to the total immunofluorescent staining in the hippocampal region of the same mouse. Total immunofluorescent staining of amyloid plaque deposition in a mouse brain can also be compared relative to the total immunofluorescent staining of amyloid plaque deposition in a control mouse brain.

In some embodiments, in a wild-derived humanized mouse model of the present disclosure, the amyloid plaque deposition in the hippocampal region may be increased by at least 5%, at least 10%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to the amyloid plaque deposition in the cortical region.

In some embodiments, a wild-derived humanized mouse model of the present disclosure has more cortical plaque deposition relative to the cortical plaque deposition of a control mouse (e.g., a WSB mouse, such as a WSB.APP/PSEN1 mouse, or a C57BL6/J mouse) of the same age. As used herein, cortical plaque deposition refers to plaque deposition in the cortical region of the brain. Cortical plaque deposition may be measured using immunofluorescent staining of amyloid precursor protein in the cortical regions of a mouse brain. Immunofluorescent staining methods, which are well-known in the art, are contemplated herein. Positive staining for amyloid precursor protein, indicating cortical plaque deposition, may be present in the cortical regions of a mouse brain of the present disclosure. Total immunofluorescent staining of cortical plaque deposition in a mouse brain is compared relative to the total immunofluorescent staining of cortical plaque deposition in a control mouse brain.

In some embodiments, a wild-derived humanized mouse model of the present disclosure has greater cerebral amyloid angiopathy relative to the cerebral amyloid angiopathy of a control mouse (e.g., a WSB mouse, such as a WSB.APP/PSEN1 mouse, or a C57BL6/J mouse) of the same age. As used herein, cerebral amyloid angiopathy refers to amyloid beta-peptide deposits within small- to medium-sized blood vessels of the brain and leptomeninges.

In some embodiments, in a wild-derived humanized mouse model of the present disclosure, the cortical plaque deposition and/or cerebral amyloid angiopathy may be increased by at least 5%, at least 10%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to the cortical plaque deposition of a control mouse (e.g., a WSB mouse, such as a WSB.APP/PSEN1 mouse, or a C57BL6/J mouse) of the same age.

In some embodiments, the plaque region-specificity of a wild-derived humanized mouse model of the present disclosure is different relative to a control mouse (e.g., a WSB mouse, such as a WSB.APP/PSEN1 mouse, or a C57BL6/J mouse) of the same age. As used herein, plaque region-specificity refers to the region of the mouse brain (e.g., the cortical or hippocampal region, or both the cortical and hippocampal regions) wherein amyloid plaque deposition may occur. In humans, plaque pathology occurs first in hippocampus (i.e., plaque region-specificity in humans occurs first in the hippocampal region). The wild-derived humanized mouse model exhibits plaque region-specificity in both the cortical and hippocampal regions of the mouse brain.

In some embodiments, the neuroinflammation of a wild-derived humanized mouse model of the present disclosure is modified relative to a control mouse (e.g., a WSB mouse, such as a WSB.APP/PSEN1 mouse, or a C57BL6/J mouse) of the same age. As used herein, neuroinflammation is indicated by positive immunofluorescent staining of microglia activation and astrocyte reactivity in the brain. Microglia activation may be measured by staining brain tissue with markers of microglia. Astrocyte reactivity may be measured by staining brain tissue with markers of astrocytes. Total immunofluorescent staining of microglia activation and/or astrocyte reactivity in the mouse brain can be compared relative to the total immunofluorescent staining microglia activation and/or astrocyte reactivity in a control mouse brain.

In some embodiments, the neuroinflammation (e.g., indicated by immunofluorescent staining of astrocyte reactivity or microglia activation) of a wild-derived humanized mouse model of the present disclosure is higher relative to a control mouse (e.g., a WSB mouse, such as a WSB.APP/PSEN1 mouse, or a C57BL6/J mouse) of the same age. In some embodiments, the neuroinflammation (e.g., indicated by immunofluorescent staining of astrocyte reactivity) of a wild-derived humanized mouse model of the present disclosure may be at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% higher relative to a control mouse (e.g., a WSB mouse, such as a WSB.APP/PSEN1 mouse, or a C57BL6/J mouse) of the same age.

In some embodiments, a wild-derived humanized mouse model of the present disclosure does not develop a tumor. In some embodiments, a wild-derived humanized mouse model of the present disclosure does not have a measurable tumor burden. As used herein, a “measurable tumor burden” refers to any non-zero tumor volume value. Any mechanism for calculating tumor volume known in the art is contemplated herein.

### Methods of Use

The mouse models provided herein (e.g., the wild-derived humanized mouse model) may be used for any number of applications. In some embodiments, a wild-derived humanized mouse model of the present disclosure exhibits neurodegeneration in the brain in response to human APOE (e.g., APOE4, APOE3, or APOE2), or human amyloid beta and/or human tau. Therefore, a wild-derived humanized mouse model of the present disclosure may be used to further assess the effects of human APOE (e.g., human APOE4), or human amyloid beta and/or human tau in a more genetically diverse mouse model.

In some embodiments, a wild-derived humanized mouse model of the present disclosure may be used to test how a particular agent (e.g., therapeutic agent) or medical procedure (e.g., cell or tissue transplantation) impacts the effects of human APOE (e.g., APOE4, APOE3, or APOE2), or human amyloid beta and/or human tau as a risk factor of AD. In some embodiments, a particular agent (e.g., therapeutic agent) may be delivered to a wild-derived humanized mouse model of the present disclosure, and changes in neurodegeneration as a result of said agent may be measured as described above relative to a wild-derived humanized mouse model of the present disclosure that did not receive said agent. Changes in neurodegeneration as a result of treatment with an agent may be indicated by an increase or decrease in NEUN staining in brain regions as described above.

Non-limiting examples of agents include therapeutic agents, such as anti-cancer agents and anti-inflammatory agents, and prophylactic agents, such as vaccines.

In some embodiments, a wild-derived humanized mouse model of the present disclosure may receive a medical procedure (e.g., cell or tissue transplantation), and changes in neurodegeneration as a result of said medical procedure may be measured as described above relative to a wild-derived humanized mouse model of the present disclosure that did not receive said medical procedure. Changes in neurodegeneration as a result of the medical procedure (e.g., cell or tissue transplantation) may be indicated by an increase or decrease in NEUN staining in brain regions as described above.

Non-limiting examples of medical procedures include transplantation of cells (e.g., microglia) from other mouse background strains or from human origin.

In some embodiments, a wild-derived humanized mouse model of the present disclosure (e.g., the WSB.APP/PSEN1/APOE4 mouse model) may be used to evaluate an effect of an agent or medical procedure on neurodegeneration in response to human APOE (e.g., APOE4, APOE3, or APOE2), or human amyloid beta and/or human tau. Thus, provided herein are methods that comprise administering an agent or medical procedure to a mouse model, and evaluating an effect of the agent or medical procedure on neurodegeneration in response to human APOE (e.g., APOE4, APOE3, or APOE2), or human amyloid beta and/or human tau in the mouse.

Assessing an effect of an agent or medical procedure on neurodegeneration in response to human APOE (e.g., APOE4, APOE3, or APOE2), or human amyloid beta and/or human tau in a wild-derived humanized mouse model of the present disclosure (e.g., the WSB.APP/PSEN1/APOE4 mouse model) includes, for example, comparing the result of the assessment with a suitable control, such as, but not limited to, the effect of the compound on a control mouse (e.g., a WSB mouse, such as a WSB.APP/PSEN1 mouse, or a C57BL6/J mouse).

### Mouse Models

Herein, for simplicity, reference is made to “mouse” and “mouse models” (e.g., surrogates for human conditions). It should be understood that these terms, unless otherwise stated, may be used interchangeably throughout the specification to encompass “rodent” and “rodent models,” including mouse, rat and other rodent species.

It should also be understood that standard genetic nomenclature used herein provides unique identification for different rodent strains, and the strain symbol conveys basic information about the type of strain or stock used and the genetic content of that strain. Rules for symbolizing strains and stocks have been promulgated by the International Committee on Standardized Genetic Nomenclature for Mice. The rules are available on-line from the Mouse Genome Database (MGD; informatics.jax.org) and were published in print copy (Lyon et al. 1996). Strain symbols typically include a Laboratory Registration Code (Lab Code). The registry is maintained at the Institute for Laboratory Animal Research (ILAR) at the National Academy of Sciences, Washington, D.C. Lab Codes may be obtained electronically at ILAR's web site (nationalacademies.org/ilar/institute-for-laboratory-animal-research). See also Davisson MT, Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.

The mouse models provide herein are transgenic mouse models that express a human amyloid precursor protein (APP) and a human presenilin 1 protein (PSEN1). In some embodiments, the transgenic mouse models express a human apolipoprotein E4 (APOE4). In some embodiments, the transgenic mouse models express a human amyloid beta. In some embodiments, the transgenic mouse models express a human tau. A transgenic mouse is a mouse having an exogenous nucleic acid (e.g., transgene) in (integrated into) its genome. Methods of producing transgenic mice are well-known.

Three conventional methods used for the production of transgenic mice include DNA microinjection (Gordon and Ruddle, Science 1981: 214: 1244-124, incorporated herein by reference), embryonic stem cell-mediated gene transfer (Gossler et al., Proc. Natl. Acad. Sci. 1986, 83:9065-9069, incorporated herein by reference) and retrovirus-mediated gene transfer (Jaenisch, Proc. Natl. Acad. Sci. 1976, 73:1260-1264, incorporated herein by reference), any of which may be used as provided herein. Genomic editing methods using, for example, clustered regularly interspace palindromic repeats (CRISPR/Cas) nucleases, transcription activator-like effector nucleases (TALENs), or zinc finger nucleases (ZFNs) are described elsewhere herein.

Following delivery of nucleic acids to a fertilized embryo (e.g., a single-cell embryo (e.g., a zygote) or a multi-cell embryo (e.g., a developmental stage following a zygote, such as a blastocyst), the fertilized embryo is transferred to a pseudopregnant female, which subsequently gives birth to offspring. The presence or absence of a nucleic acid encoding human FcRn and/or a chimeric IgG antibody may be confirmed, for example, using any number of genotyping methods (e.g., sequencing and/or genomic PCR).

New mouse models can also be created by breeding parental lines, as described in the Examples herein. With the variety of available mutant, knock-out, knock-in, transgenic, Cre-lox, Tet-inducible system, and other mouse strains, multiple mutations and transgenes may be combined to generate new mouse models. Multiple mouse strains may be bred together to generate double, triple, or even quadruple and higher multiple mutant/transgenic mice.

In some embodiments, parental mice are bred to produce F1 mice. A parental mouse may be, for example, homozygous, heterozygous, hemizygous, or homozygous null at a particular allele. Homozygous describes a genotype of two identical alleles at a given locus, heterozygous describes a genotype of two different alleles at a locus, hemizygous describes a genotype consisting of only a single copy of a particular gene in an otherwise diploid organism, and homozygous null refers to an otherwise-diploid organism in which both copies of the gene are missing.

In some embodiments of the present disclosure, one or more cells may be isolated from a mouse described by the present disclosure. In some embodiments, one or more cells isolated from a mouse of the present disclosure comprise the same genotype of a cell from said mouse.

In some embodiments, a WSB.APOE4 mouse is generated by backcrossing APOE4 onto the WSB background for multiple (e.g., 5) generations. Methods comprising propagating the progeny mice are also contemplated.

In some embodiments, a WSB.APP/PSEN1/APOE4 mouse is generated by backcrossing APOE4 onto the WSB background for multiple (e.g., 5) generations. Progeny WSB.APOE4 mice are then intercrossed with congenic WSB.APP/PSEN1 mice. The progeny offspring are intercrossed to allow for the generation of WSB.APP/PSEN1/APOE4 genotypes. Methods comprising propagating the progeny mice are also contemplated.

### Wild-Derived Humanized Mouse Models

Provided herein, in some embodiments, are wild-derived humanized mouse models. As is known in the art, wild-derived mice have genetically heterogeneous backgrounds. The introduction of transgenic mutations on genetically diverse backgrounds of wild-derived mice may be more representative of humans and may recapitulate human pathologies in a more relevant manner. Non-limiting examples of wild-derived humanized mouse models include the following mouse strains: WSB/EiJ, CAST/EiJ, and PWK/PhJ. Other wild-derived humanized mouse models are also contemplated herein.

In some embodiments, a wild-derived humanized mouse has the WSB/EiJ genotype (e.g., Jackson Labs Stock No.: #001145). The Watkins Star Line B (WSB) was derived from wild mice trapped in Eastern Shore, Maryland. Wild-derived mice are genetically distinct from common laboratory mice for a number of complex phenotypic characteristics and are valuable tools for genetic mapping, evolution and systematics research. Other wild-derived humanized mouse strains are contemplated herein.

In some embodiments, a wild-derived humanized mouse has the PWK/PhJ genotype (e.g., Jackson Labs Stock No.: #003715).

In some embodiments, a wild-derived humanized mouse has the CAST/EiJ genotype (e.g., Jackson Labs Stock No.: #000928).

### Nucleic Acids: Engineering and Delivery

The mouse models described herein comprises a nucleic acid encoding a human APP and, in some embodiments, a nucleic acid encoding a mutated human PSEN1. In some embodiments, the mouse models described herein also comprise a mouse App allele and/or a mouse Psen1 allele. In some embodiments, the mouse models comprise a human APP transgene and a mutated human PSEN1 transgene. In some embodiments, a transgene, such as a human APP transgene, and/or a mutated human PSEN1 transgene, is integrated into a mouse genome. Human APP and mutated human PSEN1 transgenes are described (JAX Stock No. 025970) and incorporated by reference herein. In some embodiments, the mouse models comprise a human apolipoprotein E (e.g., human APOE4, APOE3, or APOE2). In some embodiments, the mouse models comprise a human amyloid beta and a human tau.

The nucleic acids provided herein, in some embodiments, are engineered. An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences).

A mouse comprising a human gene is considered to comprise a human transgene. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene).

A promoter is a nucleotide sequence to which RNA polymerase binds to initial transcription (e.g., ATG). Promoters are typically located directly upstream from (at the 5′ end of) a transcription initiation site. In some embodiments, a promoter is an endogenous promoter. An endogenous promoter is a promoter that naturally occurs in that host animal.

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

An exon is a region of a gene that codes for amino acids. An intron (and other non-coding DNA) is a region of a gene that does not code for amino acids.

A nucleotide sequence encoding a product (e.g., protein), in some embodiments, has a length of 200 base pairs (bp) to 100 kilobases (kb). The nucleotide sequence, in some embodiments, has a length of at least 10 kb. For example, the nucleotide sequence may have a length of at least 15 kb, at least 20 kb, at least 25 kb, at least 30 kb, or at least 35 kb. In some embodiments, the nucleotide sequence has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb.

Any one of the nucleic acids provided herein may have a length of 200 bp to 500 kb, 200 bp to 250 kb, or 200 bp to 100 kb. A nucleic acid, in some embodiments, has a length of at least 10 kb. For example, a nucleic acid may have a length of at least 15 kb, at least 20 kb, at least 25 kb, at least 30 kb, at least 35 kb, at least 50 kb, at least 100 kb, at least 200 kb, at least 300 kb, at least 400 kb, or at least 500 kb. In some embodiments, a nucleic acid has a length of 10 to 500 kb, 20 to 400 kb, 10 to 300 kb, 10 to 200 kb, or 10 to 100 kb. In some embodiments, a nucleic acid has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb. A nucleic acid may be circular or linear.

The nucleic acids described herein, in some embodiments, include a modification. A modification, with respect to a nucleic acid, is any manipulation of the nucleic acid, relative to the corresponding wild-type nucleic acid (e.g., the naturally-occurring nucleic acid). A genomic modification is thus any manipulation of a nucleic acid in a genome (e.g., in a coding region, non-coding region, and/or regulatory region), relative to the corresponding wild-type nucleic acid (e.g., the naturally-occurring (unmodified) nucleic acid) in the genome. Non-limiting examples of nucleic acid (e.g., genomic) modifications include deletions, insertions, “indels” (deletion and insertion), and substitutions (e.g., point mutations). In some embodiments, a deletion, insertion, indel, or other modification in a gene results in a frameshift mutation such that the gene no longer encodes a functional product (e.g., protein). Modifications also include chemical modifications, for example, chemical modifications of at least one nucleobase. Methods of nucleic acid modification, for example, those that result in gene inactivation, are known and include, without limitation, RNA interference, chemical modification, and gene editing (e.g., using recombinases or other programmable nuclease systems, e.g., CRISPR/Cas, TALENs, and/or ZFNs).

A loss-of-function mutation, as is known in the art, results in a gene product with little or no function. A null mutation, which is a type of loss-of-function mutation, results in a gene product with no function. In some embodiments, an inactivated allele is a null allele. Other examples of loss-of-function mutations includes missense mutations and frameshift mutations.

A nucleic acid, such as an allele or alleles of a gene, may be modified such that it does not produce a detectable level of a functional gene product (e.g., a functional protein). Thus, an inactivated allele is an allele that does not produce a detectable level of a functional gene product (e.g., a functional protein). A detectable level of a protein is any level of protein detected using a standard protein detection assay, such as flow cytometry and/or an ELISA. In some embodiments, an inactivated allele is not transcribed. In some embodiments, an inactivated allele does not encode a functional protein.

Vectors used for delivery of a nucleic acid include minicircles, plasmids, bacterial artificial chromosomes (BACs), and yeast artificial chromosomes. It should be understood, however, that a vector may not be needed. For example, a circularized or linearized nucleic acid may be delivered to an embryo without its vector backbone. Vector backbones are small (˜ 4 kb), while donor DNA to be circularized can range from >100 bp to 50 kb, for example.

Methods for delivering nucleic acids to mouse embryos for the production of transgenic mice include, but are not limited to, electroporation (see, e.g., Wang W et al. J Genet Genomics 2016; 43 (5): 319-27; WO 2016/054032; and WO 2017/124086, each of which is incorporated herein by reference), DNA microinjection (see, e.g., Gordon and Ruddle, Science 1981:214: 1244-124, incorporated herein by reference), embryonic stem cell-mediated gene transfer (see, e.g., Gossler et al., Proc. Natl. Acad. Sci. 1986; 83:9065-9069, incorporated herein by reference), and retrovirus-mediated gene transfer (see, e.g., Jaenisch, Proc. Natl. Acad. Sci. 1976; 73:1260-1264, incorporated herein by reference), any of which may be used as provided herein.

### EXAMPLES

### Example 1. Generation of WSB.APP/PSEN1/APOE4 Mouse Model

WSB.APP/PSEN1/APOE4 were generated by first backcrossing APOE4 onto the WSB background for 5 generations. Progeny WSB.APOE4 mice were then intercrossed with congenic WSB.APP/PSEN1 mice. The progeny offspring were intercrossed to allow for the WT, APOE4 and APOE4.APP/PSEN1 genotypes.

### Immunohistochemistry

WSB, WSB.APOE4, WSB.APP/PSEN1, and WSB.APP/PSEN1/APOE4 female mice were aged to 8 months, sacrificed, and brain tissues were collected for immunohistochemistry. Brain tissues were stained with DAPI and the antibody NEUN on multiple coronal brain sections ranging from anterior, mid and posterior areas of the brain. Stained sections were then scanned with the Versa slide scanner, and the images were converted for use within IMARIS software. Cortical and hippocampal regions were traced and then the amount of NEUN corresponding with DAPI was assessed. Surface area was also measured in each of the regions traced. Representative images of 8-month female WSB of all genotypes of posterior brain regions are shown in FIG. 1A. The top graph shown in FIG. 1B depicts the amount of NEUN measured in the traced cortical regions averaged across each genotype. WSB.APOE4 have significantly less NEUN, which is a surrogate for neurons, relative to WT WSB. WSB.APOE4.APP/PSEN1 also have significantly less NEUN relative to WSB.APP/PSEN1. The lower graph shown in FIG. 1B is a comparison of surface area. The cortical surface area is lower in WSB. APOE4.APP/PSEN1 when compared with WSB.APP/PSEN1 and WSB.APOE4. Taken together, this suggests that the addition of APOE4 to the WSB context is a significant contributor to neurodegeneration, even in the absence of an amyloid driver such as APP/PSEN1.

Data from mice as described above aged to 4 months shows that WSB.APOE4 females have significantly more neurons than WSB, and WSB.APP/PSEN1/APOE4 have significantly more neurons than WSB.APP/PSEN1 in the mid-cortical region (FIG. 2). This mouse data correlates with human data that suggests that APOE4 carriers have more synapses earlier in life due to less pruning during development. None of these findings are present in the B6 background when APOE4 is present.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

1. A wild-derived humanized mouse comprising in its genome a nucleic acid encoding a human amyloid precursor protein (APP), a nucleic acid encoding a mutated human presenilin 1 protein (PSEN1), and a nucleic acid encoding a human apolipoprotein E.

2. The wild-derived humanized mouse of claim 1, wherein the human apolipoprotein Eis a human apolipoprotein E4 (APOE4).

3. A wild-derived humanized mouse comprising in its genome a nucleic acid encoding a human amyloid precursor protein (APP), a nucleic acid encoding a mutated human presenilin 1 protein (PSEN1), a nucleic acid encoding a human amyloid beta, and a nucleic acid encoding a human tau.

4. The wild-derived humanized mouse of claim 1, wherein the nucleic acid encoding a human APP is a chimeric nucleic acid comprising mouse and human coding sequences.

5. The wild-derived humanized mouse of claim 4, wherein the chimeric nucleic acid comprises a human coding sequence in the A-beta domain of a mouse APP coding sequence.

6. The wild-derived humanized mouse of claim 4, wherein the chimeric nucleic acid encodes human mutations K595N and M596L, relative to a human APP comprising the amino acid sequence of SEQ ID NO: 1.

7. The wild-derived humanized mouse of claim 6, wherein the nucleic acid encoding a human APP is an APPswe trans gene.

8. The wild-derived humanized mouse of claim 1, wherein the nucleic encoding a mutated PSEN1 comprises a human PSEN1 coding sequence that comprises a deletion in exon 9.

9. The wild-derived humanized mouse of claim 8, wherein the nucleic acid encoding a mutated PSEN1 is a PSENJ de9 trans gene.

10. The wild-derived humanized mouse of claim 9, wherein the mouse comprises in its genome Tg(APPswe,PSEN1de9)85Dbo transgene insertion.

11. The wild-derived humanized mouse of claim 1, wherein the mouse expresses the human APP, the human PSEN1, and the human apolipoprotein E, optionally human APOE4.

12. (canceled)

13. The wild-derived humanized mouse of claim 1, wherein the mouse has a genetic background selected from WSB/EiJ, CAST/EiJ, and PWK/PhJ.

14. The wild-derived humanized mouse of claim 1, wherein the mouse has at least one characteristic of early-onset Alzheimer's disease.

15. The wild-derived humanized mouse of claim 14, wherein the at least one characteristic of early-onset Alzheimer's disease is selected from neurodegeneration, cognitive deficit, and increased neuroinflammation in the brain, relative to a control.

16. The wild-derived humanized mouse of claim 1, wherein the mouse does not develop a tumor or have a measurable tumor burden.

17. The wild-derived humanized mouse of a claim 1, wherein the mouse is at least a year old.

18.-20. (canceled)

21. A cell from the mouse of claim 1.

22. A mouse comprising a cell having the same genotype of a cell from the mouse of claim 1.

23. A progeny mouse of the mouse of claim 1.

24. A method comprising producing the mouse of claim 1.


SEQUENCE LISTING (3 sequences)

SEQ ID NO: 1 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 467 aa, Homo sapiens
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 3 - protein, 128 aa, Homo sapiens
MKVLWAALLVTFLAGCQAKVEQAVETEPEPELRQQTEWQSGQRWELALGRFWDYLRWVQTLSEQVAEVRAKLEEQAQQIRLQAEAFQARLKSWFEPLVEDMQRQWAGLVEKVQAAVGTSAAPVPSDNH